(19) United States
(12) Patent Application Publication
(10) Pub. No.: US 2012/0040881 A1
(43) Pub. Date: Feb. 16, 2012

(54) GRAYING-INHIBITING WASHING AGENT

(75) Inventors: Thomas Eiting, Dusseldorf (DE); Birgit Glüsen, Dusseldorf (DE)

(73) Assignee: Henkel AG & KGaA, Dusseldorf (DE)

(21) Appl. No.: 13/239,609

(22) Filed: Sep. 22, 2011

Related U.S. Application Data
(63) Continuation of application No. PCT/EP2010/053116, filed on Mar. 11, 2010.

(30) Foreign Application Priority Data
Mar. 24, 2009 (DE) 10 2009 001 813.1

(51) Int. Cl. C11D 3/60; C07D 251/50; C07D 403/12; C07D 251/44; C07D 251/42; C07D 251/70; C07D 251/48

(52) U.S. Cl. 510/299; 544/199; 544/193.1; 544/193.2; 544/209; 544/210; 544/212; 544/213

(57) ABSTRACT

The invention relates to new triazine derivatives, the use of new triazine derivatives as graying-inhibiting active substances, and washing agent compositions comprising the new triazine derivatives as graying-inhibiting active substances along with surfactant and other ingredients found in detergent compositions.

### CROSS-REFERENCE TO RELATED APPLICATIONS

This application is a continuation of PCT Application Serial No. PCT/EP2010/053116, filed on Mar. 11, 2010, which claims priority under 35 U.S.C. §119 to 10 2009 001 813.1 (DE), filed on Mar. 24, 2009. The disclosures PCT/EP2010/053116 and DE 10 2009 001 813.1 are hereby incorporated by reference in their entirety.

### FIELD OF THE INVENTION

The present invention generally relates to new triazine derivatives and a surfactant-containing washing agent that contains a triazine derivative as a graying-inhibiting active substance.

### BACKGROUND OF THE INVENTION

The purpose of graying-inhibiting agents is to keep dirt that has been detached from the fibers during textile washing suspended in the bath, preventing the dirt from redepositing onto the textile. Water-soluble colloids, usually organic in nature, are suitable for this. Such substances include size, gelatin, salts of ethersulfonic acids of starch or of cellulose, or salts of acid sulfuric acid esters of cellulose or of starch. Water-soluble polyamides containing acid groups are also suitable for this purpose. Soluble starch preparations and starch products may also be used, including degraded starch, aldehyde starches, and the like. Polyvinylpyrrolidone is also used as an inhibitor. Cellulose ethers such as carboxymethyl cellulose (Na salt), methyl cellulose, hydroxyalkyl cellulose, and mixed ethers such as methylhydroxyethyl cellulose, methylhydroxypropyl cellulose, methylcarboxymethyl cellulose, and mixtures thereof are also often used, at levels normally from 0.1 to 5 wt % based on the washing agent.

Although these cellulose ethers have good graying-inhibiting action, their use in water-containing liquid washing agents is narrowly limited. The reason is that in addition to their graying-inhibitor effect that is relevant only in the context of use in washing methods, these cellulose ethers have comparatively low solubility in surfactant-containing systems, and have a pronounced thickening effect on aqueous systems. When they are incorporated into water-containing and in particular anionic-surfactant-containing liquid washing agents at the concentrations desired for graying-inhibiting action, either the products obtained are generally no longer flowable and pourable and can be made usable for the consumer only with additional effort, for example preparation in individual dispensing portions packaged in water-soluble or tear-open water-insoluble fashion; or the cellulose ethers, especially after storage, are not completely dissolved in the water-containing liquid washing agent, which results not only in an inadequate aesthetic impression but also in nonuniform dispensing of the graying-inhibitor active substance when the agent containing it is used. With these limitations in mind, there continues to be a need for new graying-inhibiting active materials that are compatible in water-containing, and in particular anionic-surfactant-containing, liquid washing agent compositions.

### SUMMARY OF THE INVENTION

It has now been surprisingly found that a good graying-inhibiting effect can be obtained in washing agents when specific triazine derivatives are used as the active substance. The present invention is particularly applicable to water-containing liquid washing agents, where no unreasonable viscosity increases to the washing agent or any precipitation was seen.

The subject matter of the invention is the use of triazine derivatives of the general formulas I, II, III, or IV:

T(NH—Z(SO₃M)ₐ)_{b}Y_{3-b} (I)

X(T(NH—Z(SO₃M)_{c})_{d}Y_{2-d})₂ (II)

X(T(NH—Z(SO₃M)ₑ-NH-T(NH—Z(SO₃M)_{f})Y)Y)₂ (III)

X(T(NH—Z(SO₃M)_{g}-NH-T(NH—Z(SO₃M)ₕ)₂)ᵢ(NH—Z(SO₃M)₂₋ᵢ)₂ (IV)

in which,
T denotes a 1,3,5-triazinyl residue;
Z denotes a naphthalene or benzene grouping or a straight-chain, branched, or cyclic, saturated or mono- or polyethylenically unsaturated hydrocarbon residue having 1 to 12 carbon atoms;
M denotes H, Na, Li, or K;
X denotes a linear or branched diaminoalkane, optionally interrupted by NH groups, having 1 to 20, and in particular 2 to 12 carbon atoms, or an optionally mono- or poly-SO₃M-substituted diaminostilbene, diaminobiphenyl, diaminobenzene, or piperazine grouping;
Y denotes hydrogen, chlorine, bromine, or iodine;
a denotes 1, 2, or 3;
b denotes 1, 2, or 3;
c, e, f, g, h, and k, mutually independently, denote 0, 1, 2, or 3;
d denotes 1 or 2;
i denotes 0, 1, or 2; and
the residues bound via nitrogen atoms, as well as the —Y— substituents, are located in the 2-, 4-, and 6-positions of the triazinyl ring,
wherein said triazine derivatives improve graying inhibition when used in the washing of textile fabrics.

A further subject of the invention is a washing agent, in particular an aqueous liquid washing agent, comprising surfactant as well as optional ingredients typical of washing and cleaning agents, wherein the washing agent comprises a graying-inhibiting triazine derivative of the above-defined general formulas I, II, III, or IV.

### DETAILED DESCRIPTION OF THE INVENTION

Triazine derivatives of the general formula I are obtainable by reactions of 2-halo-1,3,5-triazines with one equivalent, 2,4-dihalo-1,3,5-triazines with one or two equivalents, or 2,4,6-trihalo-1,3,5-triazines with one, two, or three equivalents of an aminoaryl or aminoalkyl compound, the aryl group of the aminoaryl compound being a benzene or naphthalene unit substituted 0 to 3 times with sulfonate. Appropriate aminoaryl compounds are, for example, 2-aminobenzenesulfonic acid, 3-aminobenzenesulfonic acid, 4-aminobenzenesulfonic acid, 2-amino-1,3-benzenedisulfonic acid, 4-amino-1,3-benzenedisulfonic acid, 2-amino-1,3,5-benzenetrisulfonic acid, 2-amino-1-naphthalenesulfonic acid, 3-amino-1-naphthalenedisulfonic acid, 4-amino-1-naphthalenesulfonic acid, 2-amino-1,5-naphthalenedisulfonic acid, 7-amino-1,6-naphthalenedisulfonic acid, 2-amino-3,6,8-naphthalenetrisulfonic acid, and 7-amino-1,3,6-naphthalenetrisulfonic acid, the sulfonic acid groups of which can also be present in salt form. The aminoalkyl compounds contain one to 12 carbon atoms; they can be straight-chain, cyclic, or branched-chain, as well as saturated or unsaturated, and are substituted at least once with sulfonate. Appropriate aminoalkyl compounds are, for example, 1-aminohexanesulfonic acid, 3-aminopentanesulfonic acid, and 2-aminobutanesulfonic acid, the sulfonic acid groups of which can also be present in salt form. Mixtures of the aforesaid aminoaryl compounds and/or of the aforesaid aminoalkyl compounds can also be used.

A reaction of two equivalents of compounds according to formula (I) that carry one or two halogens on the triazine residue, with one equivalent of C₁₋₂₀ diaminoalkane, diaminostilbene, diaminobiphenyl, diaminobenzene, or piperazine (X), yields compounds according to formula (II). Among the diaminoalkanes, α,ω-diaminoalkanes are particularly preferred, but oligo- and/or polyethyleneimines or -propyleneimines can also be appropriate; preferred oligo- and/or polyethyleneimines are those of the formula NH₂—CH₂CH₂—(NH—CH₂CH₂—)ₙNH₂, in which n is a number from 1 to 9, in particular 2 to 5; mixtures of oligo- and/or polyethyleneimines of different degrees of oligomerization and/or polymerization can also be used, so that n constitutes an average value that can also assume non-integer values. It is preferred if the diaminostilbene, diaminobiphenyl, diaminobenzene, and/or piperazine also additionally comprises at least one, in particular two or three sulfonic-acid and/or sulfonic-acid-salt substituents, for example 4, 4′-diamino-2,2′-biphenyldisulfonic acid disodium salt or 4,4′-diamino-2,2′-stilbenedisulfonic acid disodium salt. In this case in particular the aryl or alkyl compound (Z), unlike the compounds of formula (I), can also be free of sulfonic-acid-salt substituents (c=0 in formula (II)). These are by preference trans-configured stilbenes, although if applicable, the cis-configured stilbenes and mixtures of cis- and trans-configured stilbenes can also be used. Mixtures of diaminoalkanes and/or diaminostilbenes with diaminobiphenyls, diaminobenzenes, and/or piperazines can also be used.

Corresponding diaminoalkanes, diaminostilbenes, diaminobiphenyls, diaminobenzenes, or piperazines, reacted with two equivalents of 2,4,6-trihalo-1,3,5-triazine, the reaction product resulting therefrom then reacted with two equivalents of a diaminoaryl or diaminoalkyl compound H₂N—Z(SO₃Na)ₑ—NH₂, the reaction product resulting therefrom in turn reacted with two equivalents of 2,4,6-trihalo-1,3,5-triazine, and then reacted with two equivalents of an aminoaryl or aminoalkyl compound H₂N—Z(SO₃Na)_{f}, yield compounds according to the general formula (III). The aminoaryl or aminoalkyl compound in this context is identical to the aminoaryl or aminoalkyl compound discussed in the context of the manufacture of the compound according to formula (I), although the sulfonate substitution can be absent if applicable and dimethylamine and diethylamine are therefore also, in particular, appropriate as an aminoalkyl compound. The diaminoaryl or diaminoalkyl compound is selected from the compounds that correspond entirely to the aminoaryl or aminoalkyl compounds in terms of backbone and additionally carry a second amino group.

A reaction of one equivalent of trihalo-1,3,5-triazine with one equivalent of an aminoaryl or aminoalkyl compound NH₂—Z(SO₃M)_{c}, a further reaction of two equivalents of the compound thus obtainable with one equivalent of an optionally mono- or poly-SO₃M-substituted diaminostilbene, diaminobiphenyl, diaminobenzene, or piperazine (X), and subsequent reaction with two equivalents of an aminoaryl compound NH₂—Z(SO₃M)_{c}, the aryl group of the aminoaryl compounds being, mutually independently in each case, a naphthalene, acetanilide, or benzene unit 0 to 3 times substituted with sulfonate and the alkyl grouping of the aminoalkyl compounds being, mutually independently in each case, a straight-chain, branched or cyclic, saturated or ethylenically mono- or polyunsaturated hydrocarbon residue having 1 to 12 carbon atoms and 0 to 3 times substituted with sulfonate, yields compounds according to formula (IV) (where i=0). If one or more of the aryl groups contains an acetamide grouping, then a reaction with alkali carbonate, for example sodium carbonate, subsequent reaction with two equivalents of a triazine derivative of the general formula (I) that carries two halogens on the triazine residue, and subsequent reaction with 2 equivalents of an aminoaryl or aminoalkyl compound, the aryl grouping of the aminoaryl compound being a naphthalene or benzene unit substituted 0 to 3 times with sulfonate and the alkyl group of the aminoalkyl compound being a straight-chain, branched or cyclic, saturated or ethylenically mono- or polyunsaturated hydrocarbon residue having 1 to 12 carbon atoms and 0 to 3 times substituted with sulfonate, yields compounds according to formula (IV) (where i=1 or 2). It is preferred if the diaminostilbene, diaminobiphenyl, diaminobenzene, and/or piperazine also additionally comprises at least one, in particular two or three sulfonic-acid and/or sulfonic-acid-salt substituents, for example 4, 4′-diamino-2,2′-biphenyldisulfonic acid disodium salt or 4,4′-diamino-2,2′-stilbenedisulfonic acid disodium salt. In this case, in particular, the aryl or alkyl compound (Z), unlike the compounds of formula (I), can also be free of sulfonic-acid-salt substituents (g, h, and/or k=0 in formula (IV)). These are by preference trans-configured stilbenes, although if applicable, the cis-configured stilbenes and mixtures thereof can also be used. Mixtures of diaminostilbenes with diaminobiphenyls, diaminobenzenes, and/or piperazines can also be used.

A washing agent according to the present invention contains by preference 0.01 wt % to 5 wt %, and in particular 0.1 wt % to 1 wt %, of the graying-inhibiting active substance described here.

A liquid washing agent according to the present invention contains, in addition to the aforesaid graying-inhibiting active substance or mixtures thereof, and surfactants to be explained in further detail below, water in quantities (based on the entire agent) of by preference up to approximately 85 wt % and in particular from 40 wt % to 75 wt %. If necessary, some of the water may be exchanged for a water-soluble solvent component. Nonaqueous solvents that can be used in liquid agents derive, for example, from the group of mono- or polyvalent alcohols, alkanolamines, or glycol ethers, provided they are miscible with water in the indicated concentration range. The solvents are by preference selected from ethanol, n- or isopropanol, the butanols, ethylene glycol, butanediol, glycerol, diethylene glycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl, ethyl, or propyl ether, dipropylene glycol monomethyl or -ethyl ether, diisopropylene glycol monomethyl or -ethyl ether, methoxy-, ethoxy-, or butoxytriglycol, 1-butoxyethoxy-2-propanol, 3-methyl-3-methoxybutanol, propylene glycol t-butyl ether, and mixtures thereof. The quantity of nonaqueous water-soluble solvent component, based on the total quantity of the washing and cleaning agent, is equal by preference to up to 15 wt %, in particular 0.5 wt % to 10 wt %.

The washing agents according to the present invention contain at least one anionic, nonionic, cationic, and/or amphoteric surfactant. The presence of anionic surfactants is preferred, while mixtures of anionic and nonionic surfactants being particularly advantageous for a variety of applications. The total surfactant content in the liquid washing agent composition is by preference in the range from 10 wt % to 60 wt %, in particular 15 wt % to 50 wt %, based in each case on the entire liquid agent.

The nonionic surfactants used are by preference alcohol alkoxylates, i.e. alkoxylated, advantageously ethoxylated, in particular primary alcohols having by preference 8 to 18 carbon atoms and an average of 1 to 12 mol ethylene oxide (EO) per mol of alcohol, in which the alcohol residue can be linear or preferably methyl-branched in the 2-position, and/or can contain mixed linear and methyl-branched residues, such as those that are usually present in oxo alcohol residues. Particularly preferred, however, are alcohol ethoxylates having linear residues made up of alcohols of natural origin having 12 to 18 carbon atoms, e.g. from coconut, palm, tallow, or oleyl alcohol, and an average of 2 to 8 EO per mol of alcohol. The preferred ethoxylated alcohols include, for example, C₁₂₋₁₄ alcohols with 3 EO, 4 EO, or 7 EO, C₉₋₁₁ alcohols with 7 EO, C₁₃₋₁₅ alcohols with 3 EO, 5 EO, 7 EO, or 8 EO, C₁₂₋₁₈ alcohols with 3 EO, 5 EO, or 7 EO, 2-propylheptan-1-ol, and mixtures thereof, such as mixtures of C₁₂₋₁₄ alcohol with 3 EO and C₁₂₋₁₈ alcohol with 5 EO. The degrees of ethoxylation indicated represent statistical averages, which can correspond to an integral number or a fractional number for a specific product. Preferred alcohol ethoxylates exhibit a restricted distribution of homologs (narrow range ethoxylates, NRE). In addition to these nonionic surfactants, fatty alcohols with more than 12 EO can also be used. Examples of these are tallow fatty alcohol with 14 EO, 25 EO, 30 EO, or 40 EO. Nonionic surfactants that contain EO and PO groups together in the molecule are also usable according to the present invention. Block copolymers having EO-PO block units or PO-EO block units, but also EO-PO-EO copolymers or PO-EO-PO copolymers, can be used in this context. Also usable are mixed alkoxylated nonionic surfactants in which EO and PO units are distributed statistically rather than in block fashion. Such products are obtainable by the simultaneous action of ethylene oxide and propylene oxide on fatty alcohols.

Also usable as nonionic surfactants are alkyl glycosides, in particular of the general formula RO(G)ₓ in which R corresponds to a primary straight-chain or methyl-branched aliphatic residue, in particular methyl-branched in the 2-position, having 8 to 22, by preference 12 to 18 carbon atoms, and G is the symbol which denotes a glycose unit having 5 or 6 carbon atoms, preferably glucose. The degree of oligomerization x, which indicates the distribution of monoglycosides and oligoglycosides, is any number between 1 and 10; x is preferably 1.2 to 1.4.

A further class of nonionic surfactants used in preferred fashion, which are used either as the only nonionic surfactant or in combination with other nonionic surfactants, are alkoxylated, preferably ethoxylated or ethoxylated and propoxylated, fatty acid alkyl esters, by preference having 1 to 4 carbon atoms in the alkyl chain, in particular fatty acid methyl esters.

Nonionic surfactants of the amine oxide type, for example N-cocalkyl-N,N-dimethylamine oxide and N-tallowalkyl-N,N-dihydroxyethylamine oxide, and the fatty acid alkanolamides, can also be suitable. The quantity of these nonionic surfactants is by preference equal to no more than that of the ethoxylated fatty alcohols, in particular no more than half thereof.

Further suitable nonionic surfactants are polyhydroxy fatty acid amides having the general structure:

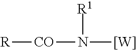

in which, R—CO denotes an aliphatic acyl residue having 6 to 22 carbon atoms; R¹ denotes hydrogen, an alkyl or hydroxyalkyl residue having 1 to 4 carbon atoms; and [W] denotes a linear or branched polyhydroxyalkyl residue having 3 to 10 carbon atoms and 3 to 10 hydroxyl groups. The polyhydroxy fatty acid amides are known substances that can usually be obtained by reductive amination of a reducing sugar with ammonia, an alkylamine, or an alkanolamine, and subsequent acylation with a fatty acid, a fatty acid alkyl ester, or a fatty acid chloride. Also belonging to the group of polyhydroxy fatty acid amides are compounds having the general formula:

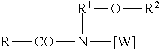

in which R denotes a linear or branched alkyl or alkenyl residue having 7 to 12 carbon atoms; R¹ denotes a linear, branched, or cyclic alkyl residue or an aryl residue having 2 to 8 carbon atoms; and R² denotes a linear, branched, or cyclic alkyl residue or an aryl residue or an oxyalkyl residue having 1 to 8 carbon atoms, C₁₋₄ alkyl or phenyl residues being preferred; and [W] denotes a linear polyhydroxyalkyl residue whose alkyl chain is substituted with at least two hydroxyl groups, or alkoxylated, preferably ethoxylated or propoxylated, derivatives of that residue. [W] is preferably obtained by reductive amination of a sugar, for example glucose, fructose, maltose, lactose, galactose, mannose, or xylose. The N-alkoxy- or N-aryloxy-substituted compounds can then be converted into the desired polyhydroxy fatty acid amides by reaction with fatty acid methyl esters in the presence of an alkoxide as catalyst.

The concentration of nonionic surfactants in the liquid washing agents is preferably 5 to 30 wt %, in particular 7 to 20 wt %, and particularly preferably 9 to 15 wt %, based in each case on the entire agent. In a preferred embodiment the nonionic surfactant is selected from alcohol alkoxylate and alkyl polyglycoside and mixtures thereof.

Anionic surfactants that can be used are, for example, those of the sulfonate and sulfate types. Possibilities as surfactants of the sulfonate type are, by preference, C₉₋₁₃ alkylbenzenesulfonates, olefinsulfonates, i.e. mixtures of alkene- and hydroxyalkanesulfonates, and disulfonates, for example such as those obtained from C₁₂-C₁₈ monoolefins having an end-located or internal double bond, by sulfonation with gaseous sulfur trioxide and subsequent alkaline or acid hydrolysis of the sulfonation products. Also suitable are alkanesulfonates that are obtained from C₁₂₋₁₈ alkanes, for example by sulfochlorination or sulfoxidation with subsequent hydrolysis and neutralization. The esters of α-sulfo fatty acids (estersulfonates), e.g. the α-sulfonated methyl esters of hydrogenated coconut, palm kernel, or tallow fatty acids, are likewise suitable.

Further suitable anionic surfactants are sulfonated fatty acid glycerol esters. “Fatty acid glycerol esters” are to be understood to comprise the mono-, di-, and triesters, and mixtures thereof, obtained during the production by esterification of a monoglycerol with 1 to 3 mol fatty acid, or upon transesterification of triglycerides with 0.3 to 2 mol glycerol. Preferred sulfonated fatty acid glycerol esters are the sulfonation products of saturated fatty acids having 6 to 22 carbon atoms, for example hexanoic acid, octanoic acid, decanoic acid, myristic acid, lauric acid, palmitic acid, stearic acid, or behenic acid.

The alkali, and in particular sodium, salts of the sulfuric acid semi-esters of the C₁₂ to C₁₈ fatty alcohols, for example from coconut fatty alcohol, tallow fatty alcohol, lauryl, myristyl, cetyl, or stearyl alcohol, or the C₁₀ to C₂₀ oxo alcohols, and those semi-esters of secondary alcohols of those chain lengths, are preferred as alk(en)yl sulfates. Additionally preferred are alk(en)yl sulfates of the aforesaid chain length that contain a synthetic straight-chain alkyl residue produced on a petrochemical basis, which possess a breakdown behavior analogous to those appropriate compounds based on fat-chemistry raw materials. For purposes of washing technology, the C₁₂ to C₁₆ alkyl sulfates and C₁₂ to C₁₅ alkyl sulfates, as well as C₁₄ to C₁₅ alkyl sulfates, are preferred. 2,3-Alkyl sulfates, such as those obtainable from Shell Oil Company under the brand DAN®, are also suitable anionic surfactants.

The sulfuric acid monoesters of the alcohol alkoxylates recited above, for example of the straight-chain or branched C₇₋₂₁ alcohols ethoxylated with 1 to 6 mol ethylene oxide, such as 2-methyl-branched C₉₋₁₁ alcohols with an average of 3.5 mol ethylene oxide (EO) or C₁₂₋₁₈ fatty alcohols with 1 to 4 EO, are also suitable.

Other suitable anionic surfactants are also the salts of alkylsulfosuccinic acid, which are also referred to as sulfosuccinates or as sulfosuccinic acid esters and represent the monoesters and/or diesters of sulfosuccinic acid with alcohols, preferably fatty alcohols, and in particular ethoxylated fatty alcohols. Preferred sulfosuccinates contain C₈₋₁₈ fatty alcohol residues or mixtures thereof. Particularly preferred sulfosuccinates contain a fatty alcohol residue that is derived from ethoxylated fatty alcohols that, considered per se, represent nonionic surfactants (see below for description). Sulfosuccinates whose fatty alcohol residues derive from ethoxylated fatty alcohols having a restricted homolog distribution are, in turn, particularly preferred. It is likewise also possible to use alk(en)ylsuccinic acid having by preference 8 to 18 carbon atoms in the alk(en)yl chain, or salts thereof.

Soaps are particularly preferred anionic surfactants. Saturated and unsaturated fatty acid soaps, such as the salts of lauric acid, myristic acid, palmitic acid, stearic acid, (hydrogenated) erucic acid and behenic acid, are suitable, as are soap mixtures derived in particular from natural fatty acids, e.g. coconut, palm-kernel, olive-oil, or tallow fatty acids. In a preferred embodiment, the washing agent contains 2 wt % to 20 wt %, in particular 3 wt % to 15 wt %, and particularly preferably 5 wt % to 10 wt % fatty acid soap. Fatty acid soaps are, in particular, an important constituent in terms of the washing power of a liquid, in particular aqueous, washing and cleaning agent. It has been surprisingly found that when the low-methylated carboxymethyl cellulose ether is used, clear and stable liquid washing agents are obtained even in the presence of a large quantity of fatty acid soap. The use of large quantities (≧2 wt %) of fatty acid soap in such systems usually results in cloudy and/or unstable products.

The anionic surfactants, including the soaps, can be present in the form of their sodium, potassium, or ammonium salts and as soluble salts of organic bases such as mono-, di-, or triethanolamine. The anionic surfactants are preferably present in the form of their sodium or potassium salts, in particular in the form of the sodium salts.

The concentration of anionic surfactants in preferred washing agents is 5 wt % to 35 wt %, in particular 8 wt % to 30 wt %, and particularly preferably 10 wt % to 25 wt %, based in each case on the entire agent. It is particularly preferred that the quantity of fatty acid soap be at least 2 wt %, particularly preferably at least 3 wt %, and in particular 4 wt % to 10 wt %. In a further preferred embodiment, the agents contain at least two, in particular three, different anionic surfactants, selected from alkylbenzenesulfonate, ether sulfate, and fatty acid soap.

The washing agent can contain a polyacrylate acting as a cobuilder and, if applicable, also acting as a thickener. Included among these polyacrylates are polyacrylate or polymethacrylate thickeners such as, for example, the high-molecular-weight homopolymers of acrylic acid crosslinked with a polyalkenyl polyether, in particular an allyl ether, of sucrose, pentaerythritol, or propylene (INCI name, according to “International Dictionary of Cosmetic Ingredients” of the Cosmetic, Toiletry and Fragrance Association (CFTA): Carbomer), which are also referred to as carboxyvinyl polymers. Polyacrylic acids of this kind are obtainable from, among other sources, the 3V Sigma company under the trade name Polygel®, e.g. Polygel DA, and from the Noveon company under the trade name Carbopol®, e.g. Carbopol 940 (molecular weight approx. 4,000,000), Carbopol 941 (molecular weight approx. 1,250,000), or Carbopol 934 (molecular weight approx. 3,000,000). Also included thereamong are the following acrylic acid copolymers: (i) copolymers of two or more monomers from the group of acrylic acid, methacrylic acid, and simple esters thereof, formed by preference with C₁₋₄ alkanols Acrylates Copolymer), included among which are, for example, the copolymers of methacrylic acid, butyl acrylate, and methyl methacrylate (CAS designation according to Chemical Abstracts Service: 25035-69-2), or of butyl acrylate and methyl methacrylate (CAS 25852-37-3), and which are obtainable, for example, from the Rohm & Haas company under the trade names Aculyn® and Acusol®, and from the Degussa (Goldschmidt) company under the trade name Tego® Polymer, e.g. the anionic nonassociative polymers Aculyn 22, Aculyn 28, Aculyn 33 (crosslinked), Acusol 810, Acusol 823, and Acusol 830 (CAS 25852-37-3); (ii) crosslinked high-molecular-weight acrylic acid copolymers, included among which are, for example, the copolymers, crosslinked with an allyl ether of sucrose or of pentaerythritol, of C₁₀₋₃₀ alkyl acrylates with one or more monomers from the group of acrylic acid, methacrylic acid, and simple esters thereof formed preferably with C₁₋₄ alkanols Acrylates/C10-30 Alkyl Acrylate Crosspolymer), and which are obtainable, for example, from the Noveon company under the trade name Carbopol®, e.g. the hydrophobized Carbopol ETD 2623 and Carbopol 1382 (INCI: Acrylates/C10-30 Alkyl Acrylate Crosspolymer), and Carbopol Aqua 30 (formerly Carbopol EX 473). Preferred washing agents contain the polyacrylate in a quantity of up to 5 wt %, in particular from 0.1 wt % to 2.5 wt %. It is advantageous if the polyacrylate is a copolymer of an unsaturated mono- or dicarboxylic acid and of one or more C₁ to C₃₀ alkyl esters of (meth)acrylic acid.

The viscosity of liquid washing and cleaning agents can be measured with usual standard methods (e.g. Brookfield LVT-II viscosimeter at 20 rpm and 20° C., spindle 3), and is by preference in the range from 500 to 5000 mPas. Preferred liquid agents have viscosities in the range from 500 mPas to 4000 mPas, values in the range from 1000 to 3500 mPas being particularly preferred.

In addition, the washing agents can contain additional ingredients that act to improve their performance and/or aesthetic properties. In the context of the present invention, preferred agents contain one or more substances from the group of the detergency builders, bleaching agents, bleach activators, enzymes, electrolytes, pH adjusting agents, fragrances, perfume carriers, fluorescing agents, dyes, hydrotropes, foam inhibitors, additional anti-redeposition agents or graying inhibitors, optical brighteners, shrinkage preventers, wrinkle protection agents, color transfer inhibitors, antimicrobial active substances, germicides, fungicides, antioxidants, corrosion inhibitors, antistatic agents, ironing adjuvants, proofing and impregnating agents, swelling and anti-slip agents, and UV absorbers.

Aluminum silicates (in particular zeolites), carbonates, salts of organic di- and polycarboxylic acids, and mixtures of these substances, may be mentioned as detergency builders that can be contained in the present agents.

The finely crystalline synthetic zeolite containing bound water that is usable is by preference zeolite A and/or zeolite P. Zeolite MAP® (commercial product of the Crosfield Co.) is particularly preferred as zeolite P. Also suitable, however, are zeolite X as well as mixtures of A, X, and/or P. Also commercially available and preferably usable in the context of the present invention is, for example, a co-crystal of zeolite X and zeolite A (approx. 80 wt % zeolite X) marketed by the Sasol company under the trade name VEGOBOND AX® and can be described by the formula:

nNa₂O.(1-n)K₂O.Al₂O₃.(2-2.5)SiO₂.(3.5-5.5)H₂O

in which n=0.90-1.0. The zeolite can be used as a spray-dried powder or also, in water-containing liquid agents, as an undried stabilized suspension still moist as manufactured. In the event the zeolite is used as a suspension, it can contain small amounts of nonionic surfactant for stabilization. For example, 1 to 3 wt % based on the zeolite, of ethoxylated C₁₂ to C₁₈ fatty alcohols with 2 to 5 ethylene oxide groups, C₁₂ to C₁₄ fatty alcohols with 4 to 5 ethylene oxide groups, or ethoxylated isotridecanols may be present in the zeolite. Suitable zeolites exhibit an average particle size of less than 10 μm (volume distribution; measurement method: Coulter Counter), and by preference contain 18 to 22 wt %, in particular 20 to 22 wt %, bound water.

The use of the generally known phosphates as builder substances is also possible, provided that such use is not restricted for environmental reasons. The sodium salts of orthophosphates, pyrophosphates, and in particular tripolyphosphates, are particularly suitable.

Suitable enzymes include those in the classes of the hydrolases, such as the proteases, esterases, lipases and/or lipolytically active enzymes, amylases, cellulases and/or other glycosyl hydrolases, and mixtures of the aforesaid enzymes. All these hydrolases contribute, in the laundry, to the removal of stains such as protein-, grease-, or starch-containing stains, and graying. Cellulases and other glycosyl hydrolases can also contribute to color retention and enhance textile softness by removing pilling and microfibrils. Oxidoreductases can also be used for bleaching and/or to inhibit color transfer. Enzymatic active substances obtained from bacterial strains or fungi, such as Bacillus subtilis, Bacillus licheniformis, Streptomyceus griseus, and Humicola insolens, are particularly suitable. Proteases of the subtilisin type, and in particular proteases obtained from Bacillus lentus, are used by preference. Enzyme mixtures, for example of protease and amylase or protease and lipase and/or lipolytically active enzymes, or protease and cellulase, or of cellulase and lipase and/or lipolytically active enzymes, or of protease, amylase, and lipase and/or lipolytically active enzymes, or protease, lipase or lipolytically active enzymes, and cellulase, but in particular protease- and/or lipase-containing mixtures and/or mixtures with lipolytically active enzymes, are of particular interest in this context. Examples of such lipolytically active enzymes are the known cutinases. Peroxidases or oxidases have also proven suitable in certain cases. Included among the suitable amylases are, in particular, α-amylases, isoamylases, pullulanases, and pectinases. Cellobiohydrolases, endoglucanases, and β-glucosidases, which are also called cellobiases, and/or mixtures thereof, are preferably used as cellulases. Because different types of cellulase differ in terms of their CMCase and avicelase activities, the desired activities can be adjusted by means of controlled mixtures of the cellulases.

The enzymes can be adsorbed onto carrier materials and/or encased in order to protect them from premature breakdown. The proportion of enzymes, liquid enzyme formulations, enzyme mixtures, or enzyme granulates can be equal to, for example, approximately 0.1 to 5 wt %, by preference 0.12 to approximately 2.5 wt %, based in each case on the entire agent.

A large number of very varied salts from the group of the inorganic salts can be used as electrolytes. Preferred cations are the alkali and alkaline-earth metals; preferred anions are the halides and sulfates. From a production-engineering standpoint, the use of NaCl or MgCl in the agents is preferred. The proportion of electrolytes in the, in particular, liquid agents is usually not more than 8 wt %, in particular 0.5 wt % to 5 wt %.

In order to bring the pH of liquid agents into the desired range, the use of pH adjusting agents may be required. All known acids and/or bases are usable here, provided their use is not prohibited for environmental or formulation reasons, and/or for reasons of consumer safety. The quantity of these pH adjusting agents usually does not exceed 10 wt % of the entire formulation.

A further component contained, if desired, in liquid agents according to the present invention is a hydrotrope. Preferred hydrotropes encompass the sulfonated hydrotropes such as, for example, the alkylarylsulfonates or alkylarylsulfonic acids. Preferred hydrotropes are selected from xylene-, toluene-, cumene-, naphthalenesulfonate or -sulfonic acid, and mixtures thereof. Counterions are by preference selected from sodium, calcium, and ammonium. If applicable, the liquid agents can encompass up to 20 wt % of a hydrotrope, in particular 0.05 wt % to 10 wt %.

In order to improve the aesthetic impression of the agents, they or at least one of their components can be colored with suitable dyes. Preferred dyes, the selection of which presents no difficulty to one skilled in the art, possess excellent shelf stability and insensitivity to the other ingredients of the agents and to light, and no pronounced substantivity with respect to textile fibers, in order not to color them.

Suitable foam inhibitors that can be used in the washing and cleaning agents are, for example, soaps, paraffin or silicone oils, which if applicable can also have been applied onto carrier materials.

Suitable anti-redeposition agents, which are also referred to as “soil repellents,” are, for example, the polymers, known from the existing art, of phthalic acid and/or terephthalic acid and/or of derivatives thereof, in particular polymers of ethylene terephthalates and/or polyethylene glycol terephthalates or anionically and/or nonionically modified derivatives thereof. Of these, the sulfonated derivatives of the phthalic acid and terephthalic acid polymers are particularly preferred.

Optical brighteners can be added to the washing and cleaning agents in order to eliminate yellowing of the treated textile fabrics. These substances absorb onto the fibers and cause brightening by converting ultraviolet radiation, which is invisible to the human eye, into longer-wave visible light, the ultraviolet light absorbed from sunlight being emitted as slightly bluish fluorescence and resulting, with the yellow tone of the yellowed laundry, in pure white. Suitable compounds derive, for example, from the substance classes of the 4,4′-diamino-2,2′-stilbenedisulfonic acids (flavonic acids), 4,4′-distyrylbiphenyls, methylumbelliferones, cumarins, dihydroquinolinones, 1,3-diarylpyrazolines, naphthalic acid imides, benzoxazole, benzisoxazole, and benzimidazole systems, and pyrene derivatives substituted with heterocycles. Optical brighteners are normally used in quantities of up to 0.5 wt %, in particular from 0.03 wt % to 0.3 wt %, based on the complete agent.

Because textile fabrics, in particular those made of rayon, viscose, cotton, and mixtures thereof, can tend to wrinkle because the individual fibers are sensitive to bending, kinking, compression, and squeezing perpendicularly to the fiber direction, the agents can contain synthetic wrinkle-prevention agents. These include, for example, synthetic products based on fatty acids, fatty acid esters, fatty acid amides, fatty acid alkylol esters, fatty acid alkylolamides, or fatty alcohols that are usually reacted with ethylene oxide, or products based on lecithin or modified phosphoric acid esters.

In order to counteract microorganisms, the washing and cleaning agents can contain antimicrobial active substances. A distinction is made here, depending on the antimicrobial spectrum and mechanism of action, between bacteriostatics and bactericides, fungistatics and fungicides, etc. Important substances from these groups are, for example, benzalkonium chlorides, alkylarylsulfonates, halogen phenols, and phenol mercuric acetate; these compounds can also be entirely dispensed with in the agents according to the present invention.

In order to prevent undesirable changes to the washing and cleaning agents and/or to the treated textile fabrics caused by the action of oxygen and other oxidative processes, the agents can contain antioxidants. This class of compounds includes, for example, substituted phenols, hydroquinones, catechols, and aromatic amines, as well as organic sulfides, polysulfides, dithiocarbamates, phosphites, and phosphonates. When such antioxidants are used, the agents according to the present invention are free of oxidizing bleaching agents.

Comfort while wearing washed textiles can result from the additional use of antistatic agents added to the washing agent compositions. Antistatic agents increase the surface conductivity and thus make possible improved dissipation of charges that have formed. External antistatic agents are usually substances having at least one hydrophilic molecule ligand, and yield a more or less hygroscopic film on the surfaces. These usually surface-active antistatic agents can be subdivided into nitrogen-containing (amines, amides, quaternary ammonium compounds), phosphorus-containing (phosphoric acid esters), and sulfur-containing antistatic agents (alkylsulfonates, alkyl sulfates). External antistatic agents are, for example, lauryl- (or stearyl)-dimethylbenzylammonium chlorides, which are suitable as antistatic agents for textile fabrics, or as an additive to washing agents for an additional textile brightening effect.

In order to improve the water absorption capability and rewettability of the treated textile fabrics and to facilitate ironing of the treated textile fabrics, silicone derivatives, for example, can be used in the washing and cleaning agents. These additionally improve the rinsing behavior of the agents thanks to their foam-inhibiting properties. Preferred silicone derivatives are, for example, polydialkyl- or alkylarylsiloxanes in which the alkyl groups comprise one to five carbon atoms and are entirely or partly fluorinated. Preferred silicones are polydimethylsiloxanes, which optionally can be derivatized and are then aminofunctional or quaternized or comprise Si—OH, Si—H, and/or Si—Cl bonds. The viscosities of the preferred silicones are in the range between 100 and 100,000 centistokes at 25° C.; the silicones can be used in quantities between 0.2 and 5 wt % based on the entire agent.

Lastly, the washing and cleaning agents can also contain UV absorbers, which are absorbed onto the treated textile fabrics and improve the light-fastness of the fibers. Compounds that exhibit these desired properties are, for example, the compounds that act by radiationless deactivation, and derivatives of benzophenone having substituents in the 2- and/or 4-position. Also suitable are substituted benzotriazoles, acrylates phenyl-substituted in the 3-position (cinnamic acid derivatives) optionally having cyano groups in the 2-position, salicylates, organic nickel complexes, and natural substances such as umbelliferone and urocanic acid.

Substances that complex heavy metals can be used in order to avoid the heavy-metal-catalyzed breakdown of certain washing-agent ingredients. Suitable heavy metal complexing agents are, for example, the alkali salts of ethylenediaminetetraacetic acid (EDTA) or of nitrilotriacetic acid (NTA), as well as alkali-metal salts of anionic polyelectrolytes such as polymaleates and polysulfonates.

A preferred class of complexing agents is the phosphonates, which are contained in preferred agents in quantities from 0.01 to 2.5 wt %, by preference 0.02 to 2 wt %, and in particular from 0.03 to 1.5 wt %. These preferred compounds include, in particular, organophosphonates such as, for example, 1-hydroxyethane-1,1-diphosphonic acid (HEDP), aminotri(methylenephosphonic acid) (ATMP), diethylenetriamine penta(methylenephosphonic acid) (DTPMP or DETPMP), and 2-phosphonobutane-1,2,4-tricarboxylic acid (PBS-AM), which are usually used in the form of their ammonium or alkali-metal salts.

The manufacture of solid agents according to the present invention presents no difficulties and can occur in known fashion, for example by spray-drying or granulation; enzymes and any further thermally sensitive ingredients, such as e.g. bleaching agents, can if applicable be added separately later on. A method comprising an extrusion step is preferred for the manufacture of agents according to the present invention having an elevated bulk weight, in particular in the range from 650 g/l to 950 g/l.

For the manufacture of agents according to the present invention in the form of tablets, which can be single-phase or multiple-phase, single-colored or multi-colored, and in particular can be made up of one layer or of multiple layers, in particular two layers, it is preferable to proceed in such a way that all the constituents (of each layer if applicable) are mixed together in a mixer, and the mixture is compressed by means of conventional tablet presses, for example eccentric presses or rotary presses, at compression pressures in the range from approximately 50 N to 100 kN, by preference at 60 to 70 kN. With multi-layer tablets in particular, it may be advantageous if at least one layer is pre-compressed. This is carried out preferably at compression pressures between 5 and 20 kN, in particular at 10 to 15 kN. Break-resistant tablets that are nevertheless sufficiently rapidly soluble under utilization conditions, having fracture strength and flexural strength values normally from 100 to 200 N but preferably above 150 N, are thereby obtained without difficulty. A tablet manufactured in this fashion preferably has a weight from 10 to 50 g, in particular from 15 g to 40 g. The tablets can have any three-dimensional shape. For example, they can be round, oval or polygonal, or any other intermediate shape. Corners and edges are advantageously rounded. Round tablets by preference have a diameter from 30 mm to 40 mm. In particular, the size of polygonal or cuboidal tablets, which are introduced predominantly via the metering apparatus of, for example, the automatic dishwasher, depends on the geometry and volume of that metering apparatus. Embodiments that are preferred by way of example have a base outline of (20 to 30 mm)×(34 to 40 mm), in particular of 26×36 mm or 24×38 mm.

Liquid respectively pasty agents according to the present invention in the form of solutions containing usual solvents are generally manufactured by simply mixing the ingredients, which can be introduced into an automatic mixer in substance or as a solution. Liquid washing agents according to the present invention are by preference clear, i.e. they exhibit no sediment and are transparent or at least translucent. The liquid washing and cleaning agents by preference have, without the addition of a dye, a transmittance for visible light (410 to 800 nm) of at least 30%, by preference at least 50%, and in particular at least 75%.

### EXAMPLES

Table 1 indicates the composition (ingredients in percent by weight, based in each case on the entire agent) of a washing agent designated “M1,” in accordance with the present invention.

TABLE 1
Exemplary washing agent composition with triazine derivative

| Ingredients (wt. percent) | M1 |
|---|---|
| C₉₋₁₃ alkylbenzenesulfonate, Na salt | 10 |
| Sodium lauryl ether sulfate with 2 EO | 5 |
| C₁₂₋₁₄ fatty alcohol with 7 EO | 10 |
| C₁₂₋₁₄ alkylpolyglycoside | 2 |
| C₁₂₋₁₆ fatty acid, Na salt | 8 |
| Glycerol | 5 |
| Trisodium citrate | 1 |
| Polyacrylate | 2 |
| Active substance | 1 |
| Enzymes, dye, opt. brightener | + |
| Water | q.s. |

The agent was tested under the following conditions:

Washing device: Atlas Launder-Ometer; 10 balls
Washing temperature: 40° C., 60 minutes
Number of washes: 5
Bath ratio: 1:12
Water hardness: 16° dH
Stain carrier: 6.7 g mixed stain (e.g. mud, dust/skin grease, soot)
Dosing ratio: 1 g of agent/200 ml

The following materials were used in the tests:

A Polyester/cotton blended fabric
B 100% cotton, pink
C 100% cotton, light yellow
D 100% cotton terrycloth, white with optical brightener
E 100% cotton, knitted cotton with optical brightener
F 100% cotton, no optical brightener

For each of the materials A-F tested, use of the active substance in accordance with the present invention resulted, as compared with a washing agent of otherwise identical composition from which the triazine derivative was absent, in an improvement in graying inhibition.

We claim:

1. A compound of formula
T(NH—Z(SO₃M)ₐ)_{b}Y_{3-b} (I);
X(T(NH—Z(SO₃M)_{c})_{d}Y_{2-d})₂ (II);
X(T(NH—Z(SO₃M)ₑ-NH-T(NH—Z(SO₃M)_{f})Y)Y)₂ (III); or
X(T(NH—Z(SO₃M)_{g}-NH-T(NH—Z(SO₃M)ₕ)₂)ᵢ(NH—Z(SO₃M)ₖ)₂₋ᵢ)₂ (IV);
wherein T represents a 1,3,5-triazinyl residue; Z represents a naphthalene or benzene moiety or a straight-chain, branched, or cyclic, saturated or mono- or polyethylenically unsaturated hydrocarbon residue having 1 to 12 carbon atoms; M represents H, Na, Li, or K; X represents a linear or branched diaminoalkane having 1 to 20 carbon atoms, or an optionally mono- or poly-SO₃M-substituted diaminostilbene, diaminobiphenyl, diaminobenzene, or piperazine moiety; Y represents hydrogen, chlorine, bromine, or iodine; a is the integer 1, 2, or 3; b is the integer 1, 2, or 3; c, e, f, g, h, and k, mutually independently, have the integer value of 0, 1, 2, or 3; d is the integer 1 or 2; i is the integer 0, 1, or 2; and wherein the residues bound via nitrogen atoms, and the —Y— substituents, are located in the 2-, 4-, and 6-positions of the triazinyl ring.

2. A compound of claim 1 wherein X, if present, represents a linear or branched diaminoalkane with 2 to 10 carbon atoms.

3. A compound of claim 1 wherein X, if present, represents a linear or branched diaminoalkane interrupted by NH groups.

4. Use of a compound of claim 1 as a graying-inhibiting active substance.

5. Use of a compound of claim 1 as a graying-inhibiting active substance, wherein said compound is formula (IV) and i represents the integer 1 or 2.

6. Washing agent composition containing as graying-inhibiting active substance a compound according to claim 1.

7. The composition of claim 6 further comprising surfactant and water, and optionally enzymes, dyes, and optical brighteners.

8. A compound of claim 1, wherein said compound has the formula (I) T(NH—Z(SO₃M)ₐ)_{b}Y_{3-b} obtained by reaction of mono-, di-, or trihalo-1,3,5-triazine with one, two, or three equivalents of an aminoaryl compound selected from 2-aminobenzenesulfonic acid, 3-aminobenzenesulfonic acid, 4-aminobenzenesulfonic acid, 2-amino-1,3-benzenedisulfonic acid, 4-amino-1,3-benzenedisulfonic acid, 2-amino-1,3,5-benzenetrisulfonic acid, 2-amino-4-sulfonic acid acetanilide, 2-amino-1-naphthalenesulfonic acid, 2-amino-1,5-naphthalenedisulfonic acid, 7-amino-1,6-naphthalenedisulfonic acid, or 7-amino-1,3,6-naphthalenetrisulfonic acid, or an aminoalkyl compound selected from 1-aminohexanesulfonic acid, 3-aminopentanesulfonic acid, and 2-aminobutanesulfonic acid, and mixtures thereof, such that the sulfonic acid groups can be present in salt form.

9. A compound of claim 1, wherein said compound has the formula (II) X(T(NH—Z(SO₃M)_{c})_{d}Y_{2-d})₂ obtained by reaction of two equivalents of a compound according to formula (I) T(NH—Z(SO₃M)ₐ)_{b}Y_{3-b} having one or two halogens on the triazine residue, with one equivalent of C₁₋₂₀ diaminoalkane, diaminostilbene, diaminobiphenyl, diaminobenzene, or piperazine.

10. The compound of claim 9, wherein X represents an α,ω-diaminoalkane.

11. The compound of claim 9, wherein X represents a diaminoalkane of formula NH₂—CH₂—CH₂—(NH—CH₂CH₂)ₙNH₂, and wherein n represents an integer from 1 to 9.

12. The compound of claim 11, wherein n represents an integer from 2 to 5.

13. The compound of claim 9, wherein said diaminostilbene, diaminobiphenyl, diaminobenzene, or piperazine comprises at least one sulfonic acid substituent.

14. A compound of claim 1, wherein said compound has the formula (III) X(T(NH—Z(SO₃M)ₑ-NH-T(NH—Z(SO₃M)_{f})Y)Y)₂ obtained by reaction of a diaminostilbene or diaminobiphenyl with two equivalents of 2,4,6-trihalo-1,3,5-triazine, followed by reaction with two equivalents of a diaminoaryl or diaminoalkyl compound of formula H₂N—Z(SO₃Na)ₑ—NH₂, followed by reaction with two equivalents of 2,4,6-trihalo-1,3,5-triazine, followed by reaction with two equivalents of an aminoaryl or aminoalkyl compound of general structure H₂N—Z(SO₃Na)_{f}.

15. A compound of claim 1, wherein said compound has the formula (IV) X(T(NH—Z(SO₃M)_{g}-NH-T(NH—Z(SO₃M)ₕ)₂)ᵢ(NH—Z(SO₃M)ₖ)₂₋ᵢ)₂ with i=0, obtained by reaction of one equivalent of trihalo-1,3,5-triazine with one equivalent of an aminoaryl compound of formula NH₂—Z(SO₃M)_{c}, followed by reaction of two equivalents of the reaction product with one equivalent of an optionally mono- or poly-SO₃M-substituted diaminostilbene, diaminobiphenyl, diaminobenzene, or piperazine, and then followed by reaction with two equivalents of an aminoaryl compound of formula NH₂—Z(SO₃M)_{c}, wherein the aryl group of the aminoaryl compounds being, mutually independently in each case, a naphthalene, acetanilide, or benzene unit 0 to 3 times substituted with sulfonate.
